(19) United States
(12) Patent Application Publication
(10) Pub. No.: US 2002/0068367 A1
(43) Pub. Date: Jun. 6, 2002

(54) Linker and method for solid phase combinatorial synthesis

(76) Inventors: David L. Coffen, San Diego, CA (US); Riccardo Pigliucci, Rancho Santa Fe, CA (US); Xiao-Yi Xiao, San Diego, CA (US)

Correspondence Address: James W. McClain/ Colleen J. McKiernan, BROWN, MARTIN, HALLER & McCLAIN, 1660 Union Street, San Diego, CA, 92101-2926, US

(21) Appl. No.: 09/975,137

(22) Filed: Oct. 10, 2001

Related U.S. Application Data
(60) Provisional application No. 60/239,564, filed on Oct. 11, 2000.

(51) Int. Cl. G01N 33/53; G01N 33/543

(52) U.S. Cl. 436/518; 435/7.1; 435/7.5

(57) ABSTRACT

A high throughput screening method for detecting interactions between proteins, nucleic acids and small molecules comprising coating a solid support surface with a substance, such as streptavidin, that has a high affinity for a ligand, such as biotin, that may be readily attached to a library of compounds via a linker molecule. The biotin linked library members are spotted onto the stretpavidin in a pattern and screened for binding to other compounds of interest. Thus, it is possible to screen much smaller quantities of compounds than would be possible in a multiwell format. Due to the high affinity of biotin for streptavidin, there is no diffusion of the compounds on the solid support. Moreover, the method provides a high throughput, low cost screen that may be accomplished completely manually without the use of expensive fluid handling robots.

### CROSS-REFERENCES TO RELATED APPLICATIONS

This application claims the benefit of priority of U.S. provisional application Serial No. 60/239,564 filed Oct. 11, 2000 which is incorporated herein by reference in its entirety.

### BACKGROUND OF THE INVENTION

The specific affinity certain biological molecules exhibit toward other molecules has often been exploited by medical and biological technologists for use in a variety of diagnostic or screening assays. When a biological molecule exhibits a binding specificity towards another, the two molecules are essentially forming a ligand/receptor linkage. An excellent example of this kind of ligand/receptor linkage is the noncovalent interaction that occurs between the protein streptavidin and the small molecule biotin. The interaction between biotin and streptavidin has widely been utilized in DNA, RNA, and protein quantification and purification. Streptavidin is a tetrameric protein. Each of the four monomers of streptavidin binds one molecule of biotin through the formation of multiple hydrogen bonds and van der Waals interactions that, when linked, are extremely durable and difficult to separate.

In its simplest form, the affinity between biotin and streptavidin can be used in methods that entail applying a biotinylated probe to a sample and then detecting the bound probe with labeled avidin or streptavidin. These techniques are commonly used to localize antigens in cells and tissues and to detect biomolecules in immunoassays and DNA hybridization techniques. Additionally, the multiple binding sites found in streptavidin allows for a number of techniques in which unlabeled avidin or streptavidin can be used to bridge two biotinylated reagents. This bridging method is commonly used to link a biotinylated probe to a biotinylated enzyme in enzyme-linked immunohistochemical applications.

Solid-phase surface chemistry, normally used for the purification and concentration of DNA samples before introducing them into separation columns, has been adapted to take advantage of the presence of biotin bridging to construct sandwich structures of biotin-streptavidin-biotin on solid surfaces. U.S. Pat. No. 5,482,867, incorporated herein by reference, describes a method for immobilizing receptors such as antibodies or antigens or oligonucleotides on surfaces of solid substrates where the surfaces are covered with caged or unactivated binding members which comprise protecting groups capable of being removed upon application of a suitable energy source. Upon immobilization of the avidin, on predefined regions of the solid surface, incubation with a desired receptor allows for simultaneous binding of biotin attached to the solid surface and biotin attached to the receptor. The binding members are protected until a spatially-addressed, suitable source of energy is applied to the regions of the surface desired to be activated. With this method, biotinylated receptors can be immobilized on activated regions of the surface previously treated with avidin. However, while the use of caged binding members is shown to be effective for immobilizing peptides, oligonucleotides or other macromolecules to a spatially addressed solid phase, presently, no means is provided for immobilizing small molecules, such as those comprising high throughput screening libraries to the spatially addressed solid phase.

In U.S. Pat. No. 5,817,527, also incorporated herein by reference, small molecules are immobilized on a solid support via a macromolecular spacer. A protein or other macromolecule is first immobilized in an aqueous medium, and the solid support is then washed with an organic solvent. The small molecule, preferably a hapten, is coupled in an organic medium, followed by organic medium washes. The preferred macromolecule is bovine gamma globulin (BGG) and gluteraldehyde is used as a cross-linker to conjugate the hapten small molecule to the immobilized protein. With this method, the new solid phases can be used for affinity purification, immunoassays and other binding assays as well as for the selection of binders by panning procedures. While this method has been shown to be successful for the linking of covalent haptens to an immobilized protein, such as BGG or BSA, the presence of an organic medium would preclude the use of any biotin-streptavidin-type linkage as cross-linker because it would be denatured in the organic medium.

Another U.S. Pat. No. 5,976,813, incorporated herein by reference, describes a Continuous-Format High Throughput Screening (CF-HTS) using a free format assay technique. In this method, a porous matrix containing a test sample is brought into contact with a gel-like matrix containing receptor, allowing the sample to diffuse into the receptor matrix. The assay components are dispensed and mixed by homogeneous bulk handling and only the test samples need be dispensed in small amounts. However, because the test samples are only spatially fixed to the porous matrix, if the testing procedure is allowed to run too long, the test samples will run together, which would be undesirable. While this method is effective for the assay of multiple test samples, the fact that the test samples are diffused into and not chemically bound to the solid support limits the number of treatment steps the samples may be subjected to.

Thus, there exists a need for improved methods for screening large numbers of small molecule libraries by high-throughput screening techniques on array plates or other multiple member solid supports. The method should entail very few mechanical operations in the screening process as well as offer an increased mechanical gain-in-function when compared to known microtiter-plate screening methods. The present invention fulfills these and other needs.

### SUMMARY OF INVENTION

It is an advantage of the present invention to provide a screening system that entails very few mechanical operations for the screening of large combinatorial libraries. A mechanical gain-in-function of at least 1000-fold can be easily realized in comparison to microtiter plate based screening methods. The quantities of both library compounds and protein required are reduced to a level limited only by the threshold limits of the method of detection.

It is another advantage of the present invention to provide a screening system that is relatively inexpensive and disposable and where the storage and data-management of very large libraries in an array plate format require comparatively minimal storage space. Moreover, the need for preparation of daughter plates from master plates is removed, thus eliminating the need for a robotic fluid handling device.

Still another advantage of the screening system of the present invention is the potential reduction of equipment and capital investment as the throughput screening of approximately 100,000 compounds can be easily achieved with an essentially manual operation.

In an exemplary embodiment of the present invention, small molecule libraries can be synthesized in a manner such that each individual library member is conjugated with biotin through a tether, usually hydrophillic in nature. Streptavidin-coated plates made of glass, plastic, or some glass/plastic composite is spotted with the individual members of the small molecule library using an arrayer to apply micro quantities of stock solutions of the compounds to individual spots on the plate. Upon application, the compounds become affixed to the spot where the stock solution is applied, due to the affinity of biotin for streptavidin. Each plate is capable of holding upwards of a 1000 (20×50) compound array and multiple copies of each plate can be prepared for use in separate binding assays. To extrapolate, 100 different plates could be used to present a 100,000 compound library. For analysis, the identity, structure, function, etc., of each compound can be correlated to its location on the numbered plate through a look-up table or electronic means, for example, spreadsheet software or like kind of computer program.

To screen for binding interactions with a protein of interest, for example, a therapeutic target, a stock solution of that protein that is preferably linked to a detectable tag (e.g. fluorescent or radioactive) is contacted with all of the library compounds that have been applied as spots on the treated plate(s) for a period of time to allow for complete reaction. Contact of the protein of interest with the library samples can be by immersion of the treated plate in a stock solution of the protein, over spotting micro aliquots of the protein stock solution on each of the affixed library compound spots, or by other means sufficient to contact the protein of interest with the library compounds applied to the treated plate(s).

After allowing a sufficient period of time for contacting the protein solution of interest with the library compound samples, the plates are washed to remove any protein which has not bound by interaction with a member of the library. The spots to which the protein has bound can then be identified using an appropriate detector. A number of suitable detectors include antibody based methods for the detection of proteins such as enhanced chemiluminescence or directly fluorescenated or radiolabeled antibodies. If the protein is labeled with a fluorescent, radioactive or other detectable tag before exposure to the plate, it may be detected directly. Automated and manual methods for the detection of fluorescent or radioactive tags including autoradiography, fluorescence imaging and the use of automated plate readers are well known.

The observation of bound protein by the method of detection of the present invention will be associated with a specific spot or series of spots on a numbered plate. The location of this spot or series of spots in the grid pattern on the plate is used to identify the library compound which is recognized by and binds to the protein of interest. The molecules thus identified can be resynthesized in untethered form for follow-up and secondary analysis.

In a second exemplary embodiment, plates of specific compounds prepared for the purpose of characterizing gene products of unknown function can be readily prepared using the screening system of the present invention. Thus, for example, a plate of all known neurotransmitters and central nervous system (CNS) drugs known to be agonists, antagonists, or modulators of transporter proteins for neurotransmitters could be used to identify an association of protein of unknown function with the CNS. Similarly, a plate of all known hormonal peptides and their cognate drug substances could be used to establish association of a protein with the endocrine or autocrine systems. A plate of proteolytic enzyme inhibitors could help identify new proteins of that class, kinase inhibitors for signal transduction, and so on.

### DESCRIPTION OF THE DRAWINGS

Understanding the present invention will be facilitated by consideration of the following detailed description of a preferred embodiment of the present invention taken in conjunction with the accompanying drawings, in which like numerals refer to like parts in which:

FIG. 1 is an illustration of the generic System 1 and System 2 interactions between biotin and streptavidin. FIGS. 1a and 1b illustrate the generic System 1 where biotin is hydrophillicly linked to a nitrophenyl moiety and bound to streptavidin coated on solid phase. FIG. 1c represents an anti-dinitrophenyl (DNP) antibody with a bound fluorescein marker, which in turn is bound to the nitrophenyl moiety/tether/biotin/streptavidin as show in FIG. 1d. FIGS. 1e and 1f illustrate the generic System 2 where two biotin molecules are hydrophillicly linked together and bound to streptavidin coated on solid phase. FIG. 1g represents a streptavidin molecule with bound fluorescein marker, which is bound to the free biotin/tether/biotin/streptavidin as shown in FIG. 1h;

FIG. 2 illustrates the chemical composition of linkers 1-7 according to the present invention;

FIG. 3 illustrates the chemical reaction resulting in the product, linker 3;

FIG. 4 illustrates the chemical reaction resulting in the product, linker 5;

FIG. 5a is a plot of data from Table 5, derived from evaluation of linker 3. FIG. 5b lists the various reaction conditions for the assay of linker 3;

FIG. 6 is a plot of data from Table 6, derived from evaluation of linker 3;

FIG. 7 is a plot of data from Table 7, derived from evaluation of linker 3;

FIG. 8 is a plot of data from Table 8, derived from evaluation of linker 3;

FIG. 9a is a plot of fluorescence emission derived from evaluation of linker 5. FIG. 9b lists the various reaction conditions for the assay of linker 5;

FIG. 10 is a plot of data from Table 9, derived from evaluation of linker 5;

FIG. 11 is a plot of data from Table 10, derived from evaluation of linker 3; and

FIG. 12 is a plot of data from Table 11, derived from evaluation of linker 3.

### DETAILED DESCRIPTION OF THE PREFERRED EMBODIMENT

The following detailed description is addressed to a new format for presenting small-molecule libraries to protein-based high-throughput binding assays where each individual library member is conjugated with biotin through a chemical tether. As will be readily apparent to those skilled in the art, the compounds and methods described herein may be adapted for use in characterizing products of unknown function, establishing association of a given molecule or protein with a variety of biochemical systems, and is not restricted to the exemplary embodiment that follows.

The present invention provides a screening system that entails very few mechanical operations for the screening of large combinatorial libraries while enabling a mechanical gain-in-function of at least 1000-fold in comparison to microtiter-plate based screening methods. The quantities of both library compounds and sample protein required for the screening assays are reduced to a level limited only by the threshold limits of the method of detection. For example, the fluorescence emmision generated by low picomolar amounts of fluorescein is easily within the range of detection using a commercially available plate reader such as the Spectra MAX Gemini plate reader (Molecular Devices Corp., Sunnyvale, Calif.).

In addition to facilitating a more sensitive method of detection, the present invention also provides a screening system that is relatively inexpensive and disposable. In instances where there is a minimum of both physical storage space and computer memory for data-management, the array plate format of the present invention requires comparatively little space. Because the need for preparation of daughter plates from master plates is removed, the need for bulky and costly robotic fluid handling devices is eliminated, potentially reducing the equipment and capital investment normally associated with high throughput screening laboratory set-up. The screening of approximately 100,000 compounds can be easily achieved with an essentially manual operation.

Using the method of the present invention, small molecule libraries can be synthesized in a manner such that each individual library member is attached to a biotin molecule utilizing a tether, usually hydrophillic in nature, to bridge the two species. Small molecule library members can be from enzyme inhibitors, peptides, biologically active compounds, small molecules or any compound of interest that may be linked to biotin by a tether. Streptavidin-coated plates made of glass, plastic, or some glass/plastic composite, similar to those commercially available from Xenopore Corp. (Hawthorne, N.J.) are individually spotted with each of the biotinylated small molecules. A standard arrayer, such as a Genetic MicroSystems GMS™ 417 arrayer, facilitates the application of micro quantities of stock solutions of the compounds to precise and distinct areas on the plate. Due to the high affinity of biotin for streptavidin, the biotinylated compounds become affixed to the spot on the plate where the stock solution is dispensed by the arrayer. Each plate has a useable surface area capable of holding upwards of a 1000 (20×50) compound array. Furthermore, multiple copies of each plate can be easily and accurately prepared for use in separate binding assays. To extrapolate, 100 different plates can be used to present a 100,000 compound library. For analysis, the identity, structure, function, or any other relevant characteristic of each compound can be linked to its coordinates on the numbered plate by correlating its location in table or graph data or using electronic means, such as spreadsheet software or similar computer program.

To screen for binding interactions with a protein of interest, for example a therapeutic target or antibody, a homogeneous stock solution of the test protein is introduced to each of the individual library compounds that have been applied as spots on the streptavidin treated plate(s) for a period of time long enough to allow for complete reaction. Contact between the protein of interest and the individual library samples can be made by immersing the treated plate in a stock solution of the protein. Bulk treatment of the plates is possible because the individual biotinylated library compounds are sufficiently tightly bound to distinct spots on the streptavidin treated plate so that cross-diffusion of library samples does not occur. Over-spotting micro aliquots of the protein stock solution on each of the conjugated library compound spots or other means sufficient to contact the protein of interest with the library compounds bound to the treated plate(s) represent alternate methods for screening binding interactions.

After allowing a sufficient period of time for contacting the protein solution of interest with the library compound samples, the plates are washed to remove any protein which is not bound by interaction with a member of the library. The spots to which the protein is bound can then be identified using an appropriate detection means. Detectable labels include fluorescent and radioactive tags. The selection of a tag is not a limitation of the instant invention, but is instead a choice frequently made by those skilled in the art depending on other parameters of the screening. For example, a specific single fluorescent probe could be selected if the compounds of the library fluoresced in response to excitation with certain wavelengths of light. Different proteins could be tagged with different fluorescent tags with different spectra (e.g. fluorescein, rhodamine and Texas Red or a series of Alexa or BODIPY dyes from Molecular Probes) to allow for a single array of compounds to be screened with a number of proteins simultaneously. Tags may also be selected dependent upon the detectors available to the individual performing the screening assay. Fluorescent tags and absorption and emission spectra are well known to those skilled in the art. An extensive list of fluorescent tags and their corresponding spectra may be readily found in a number of sources (e.g. http://www.probes.com/servlets/spectra). Similarly, the selection of a radioactive tag is a matter of choice routinely made by those skilled in the art depending on detectors available, facilities, etc. A number of isotopes including, but not limited to ³H, ³³P, ³⁵S and ¹²⁵I can be used with the method of the invention. Alternatively, protein may be detected indirectly by antibody based screening methods including enhanced chemiluminescence, or through the use of fluorescently or radioactively labeled antibodies. The label can be detected by a number of methods well known to those skilled in the art bound to the treated plate(s) represent alternate methods for screening binding interactions.

After allowing a sufficient period of time for contacting the protein solution of interest with the library compound samples, the plates are washed to remove any protein which is not bound by interaction with a member of the library. The spots to which the protein is bound can then be identified using an appropriate detection means. Detectable labels include fluorescent and radioactive tags. The selection of a tag is not a limitation of the instant invention, but is instead a choice frequently made by those skilled in the art depending on other parameters of the screening. For example, a specific single fluorescent probe could be selected if the compounds of the library fluoresced in response to excitation with certain wavelengths of light. Different proteins could be tagged with different fluorescent tags with different spectra (e.g. fluorescein, rhodamine and Texas Red or a series of Alexa or BODIPY dyes from Molecular Probes) to allow for a single array of compounds to be screened with a number of proteins simultaneously. Tags may also be selected dependent upon the detectors available to the individual performing the screening assay. Fluorescent tags and absorption and emission spectra are well known to those skilled in the art. An extensive list of fluorescent tags and their corresponding spectra may be readily found in a number of sources (e.g. http://www.probes.com/servlets/spectra). Similarly, the selection of a radioactive tag is a matter of choice routinely made by those skilled in the art depending on detectors available, facilities, etc. A number of isotopes including, but not limited to ³H, ³³P, ³⁵S and ¹²⁵I can be used with the method of the invention. Alternatively, protein may be detected indirectly by antibody based screening methods including enhanced chemiluminescence, or through the use of fluorescently or radioactively labeled antibodies. The label can be detected by a number of methods well known to those skilled in the art including autoradiography, fluorescence imaging and by use of an automated plate reader, CCD camera or other suitable detector depending on the tag to be detected.

When a binding reaction is observed, correlating to a specific spot or series of spots on a numbered plate, the location of this spot or series of spots is located in the grid pattern on the plate which is then used to identify the specific library compound which has recognized and bound to the protein of interest. The molecules thus identified can be resynthesized in untethered form for follow-up and secondary analysis.

Using the system of the present invention, plates of specific compounds prepared for the purpose of characterizing gene products of unknown function can be readily prepared. For example, a plate of all known neurotransmitters and CNS drugs previously recognized to be agonists, antagonists, or modulators of transporter proteins for neurotransmitters can be prepared in order to identify any association between a protein of unknown function and the CNS. Similarly, a plate of all known hormonal peptides and their cognate drug substances can be used to establish an association between a sample protein and the endocrine or autocrine systems. A plate of proteolytic enzyme inhibitors could help identify new proteins of that class, kinase inhibitors for signal transduction, and so on.

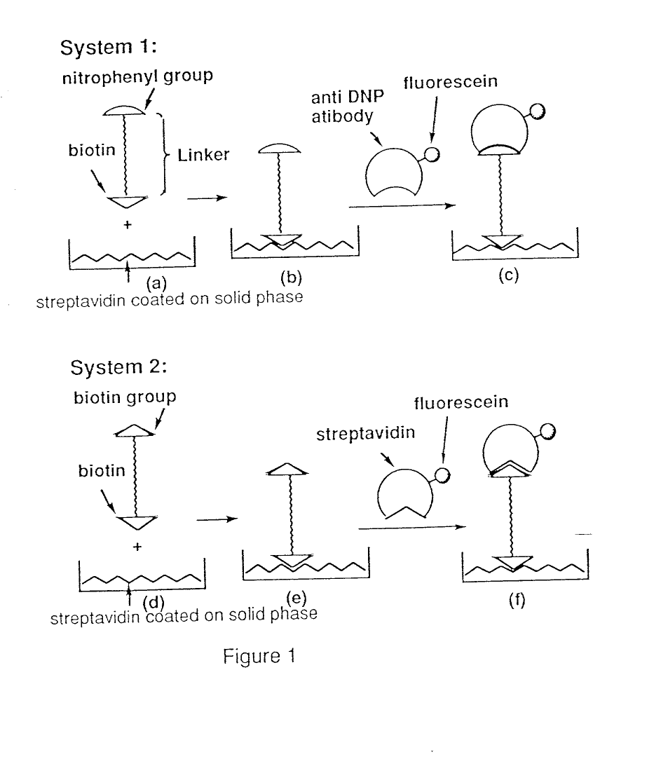

Referring to the drawings, and particularly to FIG. 1, generic reactions illustrating two variations of the screening system of the present invention are shown. FIGS. 1a-1d illustrate the generic System 1 reaction wherein a compound comprised of a tether connecting biotin with a chemical compound containing a nitrophenyl group is applied to a streptavidin coated solid phase. The amount of fluorescein-protein complex (c), where the exemplary protein is fluorescein-anti-DNP antibody, bound to the biotin-tether-nitrophenyl complex is characterized by detecting the magnitude of fluorescence emission in each individual well at the completion of the reaction. Similarly, FIG. 1e-1h illustrates the generic System 2 reaction wherein a tether connects 2 biotin groups together and streptavidin is coated on the solid phase. As in the System 1 reaction, the amount of fluorescein-protein complex (g), where the exemplary protein is fluorescein-streptavidin, bound to the biotin-tether-biotin complex is characterized through the detection of fluorescence emissions in each individual well at the completion of the reaction. In the exemplary variations of the screening system of the present invention, when a fluorescence emission signal is detected, it demonstrates that certain fluorescein-protein complexes have bound to the corresponding linkers in these assays.

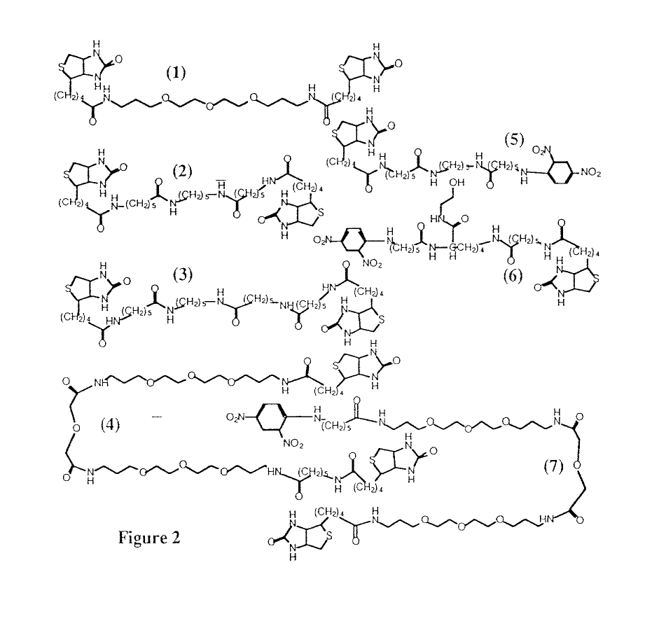

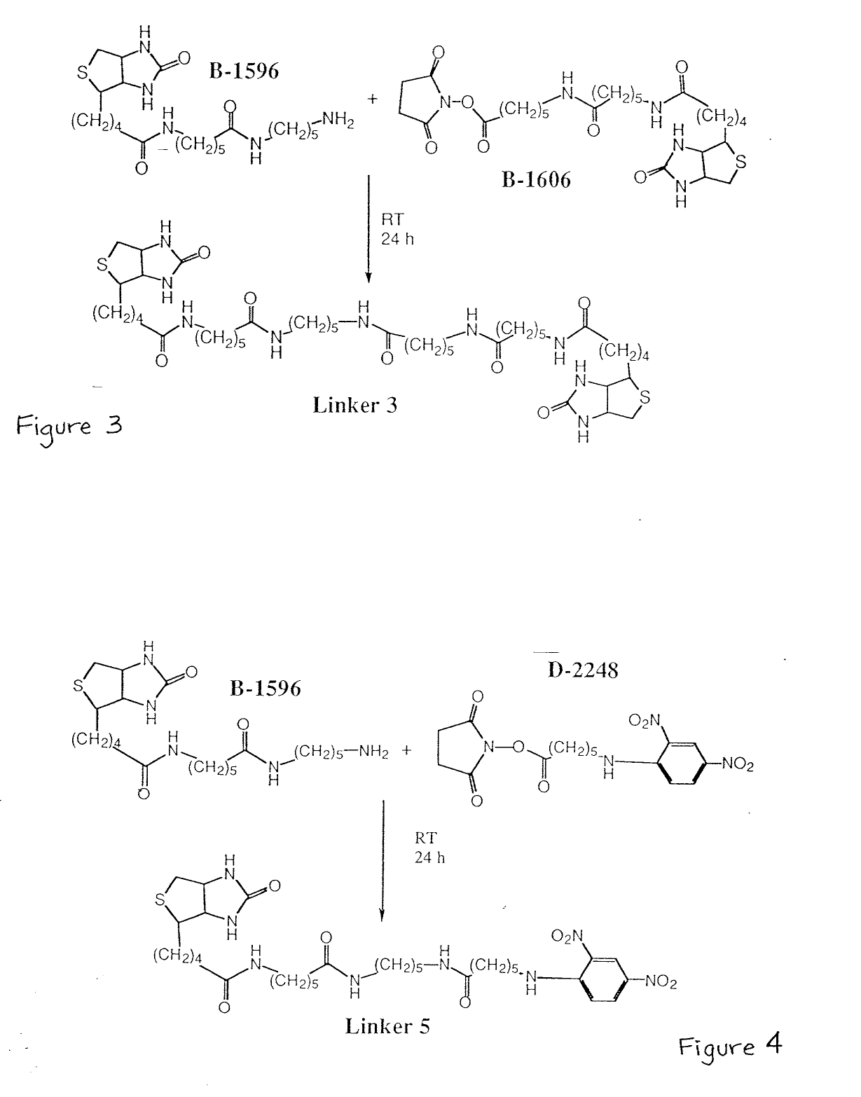

Specific examples of linkers, biotin-tether-small molecule complexes, used in the screening system of the present invention are provided in FIG. 2. The linkers are synthesized using commercially available biotinylated reagents available from Molecular Probes, Inc. of Eugene, Oreg. More specifically, amine-reactive biotinylated reagents such as certain biotin-succinimidyl esters (product #s B-1582, B-1606, and D-2248) and biotin-cadaverines (product # B-1596) act as intermediates for coupling biotin to DNA, carboxylic acids and other biomolecules. Thus, both the biotin group and the tether are provided in a single, commercially available biotinylation reagent for reaction with the small molecule of interest. Linker 2, illustrated in FIG. 2b, is prepared by combining 100 μL of 50 mM of B-1596 in DMSO with 100 μL of 50 mM of B-1582. The mixture, kept at room temperature for 24 hours, yields a product with an observed molecular weight of 781.449. Linker 3, illustrated in FIG. 2c, is similarly prepared by combining 100 μL of 50 mM of B-1596, in DMSO, with 100 μL of 50 mM of B-1606, the reaction which is shown in FIG. 3. As in the preparation of linker 2, the reaction mixture of linker 3 is maintained at room temperature for 24 hours, yielding a product with an observed molecular weight of 894.534. Linker 6, illustrated in FIG. 2f, is derived in a manner similar to linkers 2 and 3, producing a final product with an observed molecular weight of 721.00. Linker 5, illustrated in FIG. 2e, is prepared by combining 100 μL of 50 mM of B-1596, in DMSO, with 100 μL of 50 mM of D-2248, the reaction which is shown in FIG. 4. Linker 1, illustrated in FIG. 2a. and linkers 4 and 7, illustrated in FIGS. 2d and 2g, respectively, are derived from dimerizing two or more molecules of B-1582.

In the quantification of the interaction between fluoresceinated-proteins and linkers is characterized by detecting the magnitude of fluorescence emissions in each individual well at the completion of standard protein-binding assay procedures. If a fluorescence emission signal is detected, it demonstrates that certain fluoresceinated-proteins have bound to the corresponding linkers in the assay and vice versa.

A standard protein-binding assay comprises a multi-step process in which strepeavidin pre-treated 96-well plates are exposed to a solution containing linker followed by exposure to a solution containing fluorescein-labeled protein. The 96-well plates are coated with a commercially available streptavidin solution (cat #BPS00100; Xenopore Corp., Hawthorne, N.J.) and then pre-incubated with 300 μl PBS buffer for 10 minutes at room temperature. To complete the pre-incubation process, the PBS buffer is discarded and the plates are further washed five times with water. Once the plate is sufficiently washed, add to the wells 100 μl of pre-mixed binding buffer containing the desired linker, the preparation of which is as described above. After addition of the linker solution, the plate is incubated at 37° C. for one hour. Upon completion of incubation, the linker solution is discarded and the plate is washed with five changes of water. For the fifth step of the assay process, 100 μl of a premixed reaction solution containing 1 M Tris, 0.5 M NaCl and the fluorescein-labeled proteins are added to selected wells of the plate with bound linker. The fluorescein conjugated proteins, anti-dinitrophenyl antibody (rabbit IgG fraction, cat #A-6423) (anti-DNP) and streptavidin (cat #189734) are commercially available from Molecular Probes, Inc., Eugene, Oreg. and Calbiochem Inc., San Diego, Calif. Upon addition of the protein solutions, the plate is further incubated at 37° C. for one hour. Following the incubation period, the reacting solution in each well is discarded and the wells are washed with five changes of 1 M Tris or 30 changes of water. After this final washing step, 250 μl of elution buffer containing 0.5 mM NaOH, methanol and 20 mM EDTA is added to each well and the plate is read using a Spectra MAX Gemini fluorescein plate reader (Molecular Devices Corp., Sunnyvale, Calif.).

Using the standard protein-binding assay described above, linkers 1-7 are evaluated for their ability to bind fluorescein-streptavidin or fluorescein-anti-DNP. The detection of fluorescence emission beyond that of the control would indicate that the fluorescein labeled protein has bound to the corresponding linker. Lack of fluorescence emission would indicate that either the linker or the fluorescein-bound proteins are absent, or that an improper linker was used. This may be interpreted as meaning that the protein of interest, in this exemplary example streptavidin or anti-DNP, “recognizes” the particular library compound being used in the experiment. It is this “recognition” that constitutes the use of these plate libraries as “affinity probes”. While preliminary test results are reported for all of the tested linkers, particular emphasis will be on the data derived from testing of linkers 3 and 5.

Linker 1

No fluorescence emission was detected from the positive experiment, where both linker and protein were present, when linker 1 was examined.

Linkers 2, 4, 6 and 7

Fluorescence emissions were detected when these linkers were examined. The concentration of linker and protein used in each experiment and the resulting fluorescence emission for each linker are shown in Tables 1-4, below.

TABLE 1
Linker 2

| | | | | | | | |
|---|---|---|---|---|---|---|---|
| [Linker 2] | 62.5 | nmol | — | | 62.5 | nmol | — |
| [Streptavidin- | 73.5 | pmol | 73.5 | pmol | — | | — |
| fluorescein] | | | | | | | |
| Fluorescence emission | 652 | | 585 | | 583 | | 566 |

TABLE 2
Linker 4

| | | | | | | | |
|---|---|---|---|---|---|---|---|
| [Linker 4] | 5 | nmol | — | | 5 | nmol | — |
| [Streptavidin- | 100 | pmol | 100 | pmol | — | | — |
| fluorescein] | | | | | | | |
| Fluorescence emission | 218 | | 129 | | 122 | | — |

TABLE 3
Linker 6

| | | | | | | | |
|---|---|---|---|---|---|---|---|
| [Linker 6] | 50 | nmol | — | | 50 | nmol | — |
| [Streptavidin- | 30 | pmol | 30 | pmol | — | | — |
| fluorescein] | | | | | | | |
| Fluorescence emission | 729 | | 666 | | 694 | | 663 |

TABLE 4
Linker 7

| | | | | | | | |
|---|---|---|---|---|---|---|---|
| [Linker 7] | 5 | nmol | — | | 5 | nmol | — |
| [Streptavidin- | 266 | pmol | 266 | pmol | — | | — |
| fluorescein] | | | | | | | |
| Fluorescence emission | 208 | | 116 | | 112 | | — |

Linker 3

To verify that the fluorescence emission detected in the assay was not the result of a non-specific interaction between the streptavidin coated on the 96 well plates and the added fluoresceinated protein, or any other factors, 36 variable control experiments, in a 6×6 configuration were conducted including a positive control. As shown in the numerical data of Table 5 and the corresponding plot of FIG. 5, when both linker (linker 3 in this exemplary example) and fluorescein-streptavidin (Cat# 189734, Calbiochem-Novabiochem Corp., La Jolla, Calif. were present (Well #1), the detected fluorescence emission was about 500 while this parameter was less than 150 in the other control experiments where either linker or fluoresceinated protein was absent, or improper linker was used. Additionally, where a “pre-linker” (B-1596, Molecular Probes, Inc.) replaced linker 3 in the reaction, fluorescence emission remained at a level similar to reactions where no linker was used. Therefore, the higher fluorescence emission in Well #1 was caused by the fluorescein-streptavidin binding to the corresponding linkers rather than non-specific interaction between proteins, or any other factors.

TABLE 5

| | Column | Column | Column | Column | Column | Column |
|---|---|---|---|---|---|---|
| | 1 | 2 | 3 | 4 | 5 | 6 |
| Row 1 | 492.22 | 494.83 | 450.81 | 987.05 | 1437.9 | 479.29 |
| Row 2 | 138.08 | 133.87 | 128.66 | 271.95 | 400.61 | 133.54 |
| Row 3 | 134.37 | 127.45 | 127.80 | 261.82 | 389.62 | 129.87 |
| Row 4 | 129.91 | 155.23 | 108.79 | 285.14 | 393.93 | 131.31 |
| Row 5 | 127.92 | 131.07 | 128.76 | 258.99 | 387.75 | 129.25 |
| Row 6 | 157.89 | 104.48 | 119.34 | 262.37 | 381.71 | 127.24 |

The amount of linker #3 and “pre-linker”, B-1596, used per reaction was 50 nmol each. 100 pmol of the streptavidin-fluorescein is used per reaction.

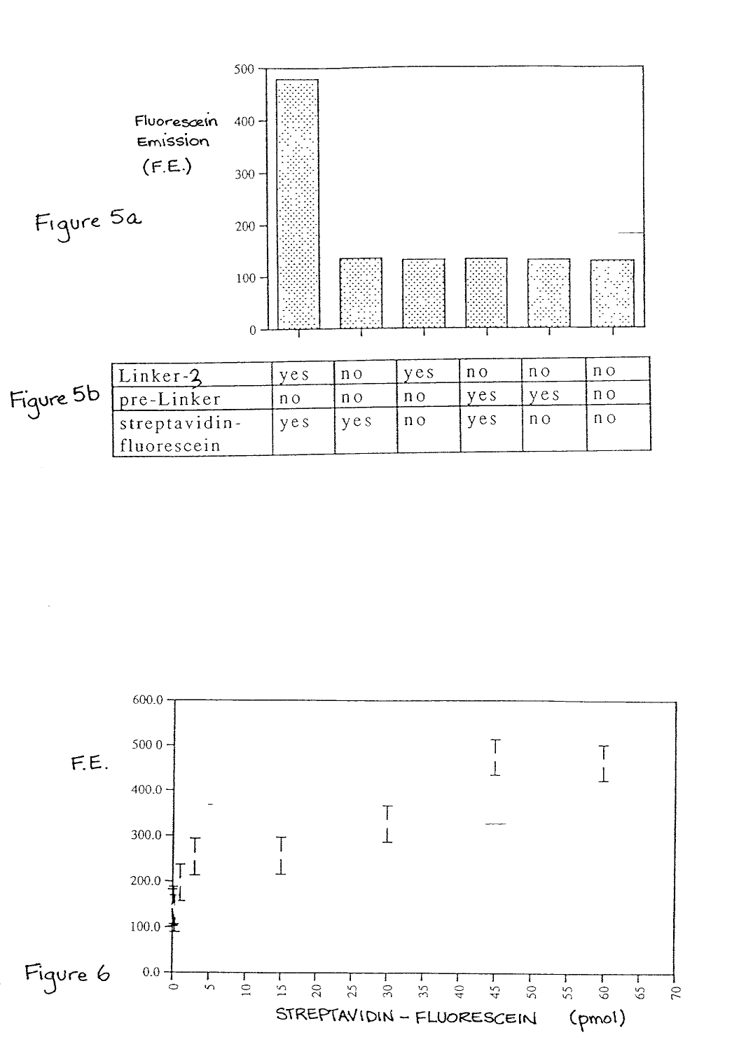

As shown by the numerical data in Table 6 and the graph of the corresponding FIG. 6, the concentration of fluorescein-streptavidin was examined while the concentration of linker was maintained at a constant 50 nmol. A total of 60 experiments were run with nine different concentrations of fluorescein-streptavidin, ranging from 0 to 60 pmol.

TABLE 6

| | | | | | | | [STREP] |
|---|---|---|---|---|---|---|---|
| | Column 1 | Column 2 | Column 3 | Column 4 | Column 5 | Column 6 | pmol |
| Row 1 | 548.75 | 410.38 | 431.15 | 959.13 | 1390.3 | 463.43 | 60.0 |
| Row 2 | 454.46 | 540.03 | 428.66 | 994.49 | 1423.2 | 474.38 | 45.0 |
| Row 3 | 395.67 | 206.13 | 376.78 | 601.80 | 978.58 | 326.19 | 30.0 |
| Row 4 | 220.15 | 262.13 | 288.79 | 482.28 | 771.07 | 257.02 | 15.0 |
| Row 5 | 237.61 | 304.43 | 218.74 | 542.04 | 760.78 | 253.59 | 3.0 |
| Row 6 | 167.36 | 221.92 | 202.19 | 389.28 | 591.47 | 197.16 | 1.0 |
| Row 7 | 72.275 | 154.69 | 162.22 | 226.97 | 389.19 | 129.73 | 0.3 |
| Row 8 | 110.57 | 165.87 | 166.59 | 276.44 | 443.03 | 147.68 | 0.15 |
| Row 9 | 158.28 | 152.84 | 138.89 | 311.12 | 450.01 | 150.00 | 0 |
| Row 10 | 156.28 | 124.02 | 147.16 | 280.3 | 427.46 | 142.49 | 0 |

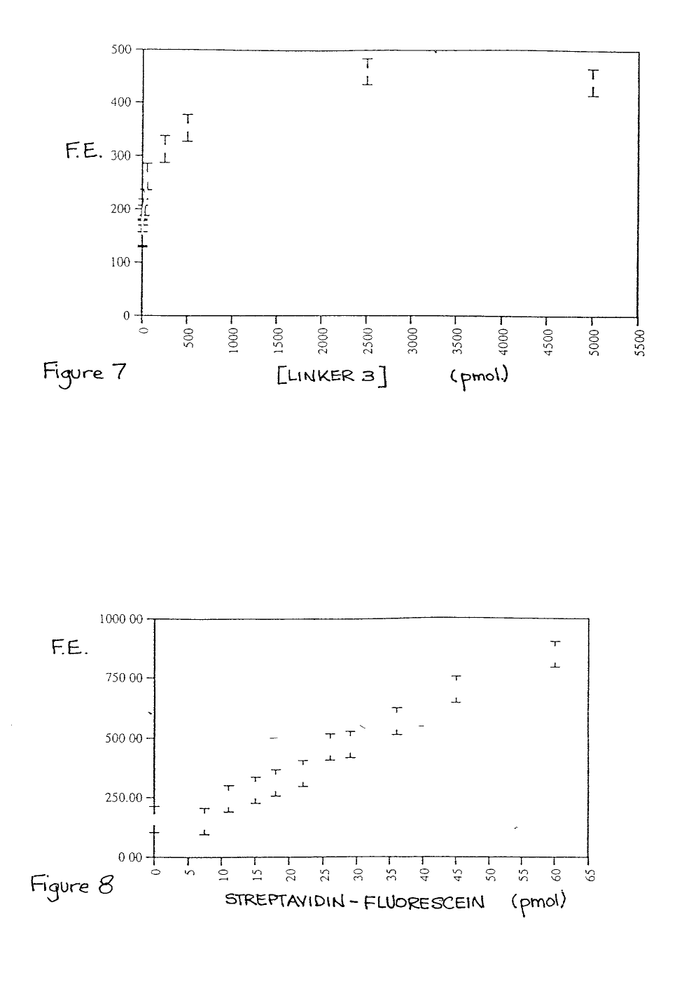

Similarly, the concentration dependence of linker 3 was examined while the concentration of fluorescein-streptavidin (100 pmol) was kept unchanged as shown by the data in Table 7 and the plot of the corresponding FIG. 7. A total of 60 experiments were run with nine different concentrations of the linker #3, ranging from 0 to 5000 nmol.

TABLE 7

| | | | | | | | [Linker] |
|---|---|---|---|---|---|---|---|
| | Column 1 | Column 2 | Column 3 | Column 4 | Column 5 | Column 6 | nmol |
| Row 1 | 447.77 | 435.80 | 437.09 | 883.57 | 1320.7 | 440.22 | 5000.0 |
| Row 2 | 474.19 | 446.49 | 452.76 | 920.68 | 1373.4 | 457.81 | 2500.0 |
| Row 3 | 325.94 | 377.74 | 354.91 | 703.68 | 1058.6 | 352.86 | 500.0 |
| Row 4 | 261.21 | 336.44 | 342.82 | 597.65 | 940.47 | 313.49 | 250.0 |
| Row 5 | 226.70 | 275.81 | 280.45 | 502.51 | 782.96 | 280.99 | 50.0 |
| Row 6 | 223.84 | 245.68 | 166.21 | 469.52 | 635.73 | 211.91 | 25.0 |
| Row 7 | 239.14 | 227.96 | 114.31 | 467.1 | 581.41 | 193.80 | 5.0 |
| Row 8 | 230.68 | 175.46 | 137.77 | 406.14 | 543.91 | 181.30 | 2.5 |
| Row 9 | 182.28 | 171.25 | 112.70 | 353.53 | 466.23 | 155.41 | 0 |
| Row 10 | 159.28 | 166.93 | 130.65 | 326.21 | 456.86 | 152.29 | 0 |

Table 8 shows a listing of the numerical data and the corresponding FIG. 8 illustrates the resulting fluorescence emission standard curve which can be used to approximate the amount of fluorescein-protein binding to the linkers.

TABLE 8

| | | | | | | | [ST/FL] |
|---|---|---|---|---|---|---|---|
| | Column 1 | Column 2 | Column 3 | Column 4 | Column 5 | Column 6 | pmol |
| Row 1 | 837.57 | 816.34 | 889.88 | 1651.9 | 2541.8 | 847.26 | 60.0 |
| Row 2 | 688.65 | 685.98 | 717.4 | 1374.6 | 2092.0 | 697.34 | 45.0 |
| Row 3 | 569.16 | 571.94 | 561.36 | 1141.1 | 1702.5 | 567.49 | 36.0 |
| Row 4 | 495.93 | 505.81 | 415.18 | 1001.7 | 1416.9 | 472.31 | 29.0 |
| Row 5 | 496.71 | 447.37 | 441.48 | 944.08 | 1385.6 | 461.85 | 26.0 |
| Row 6 | 363.91 | 359.81 | 327.71 | 723.72 | 1051.4 | 350.48 | 22.0 |
| Row 7 | 361.66 | 271.35 | 299.99 | 633.01 | 933.0 | 311.00 | 18.0 |
| Row 8 | 307.82 | 258.6 | 278.43 | 566.42 | 844.85 | 281.62 | 15.0 |
| Row 9 | 270.54 | 227.73 | 239.01 | 498.27 | 737.28 | 245.76 | 11.0 |
| Row 10 | 112.81 | 170.99 | 167.3 | 283.8 | 451.1 | 150.37 | 7.4 |
| Row 11 | 126.88 | 172.89 | 173.4 | 299.77 | 473.17 | 157.72 | 0 |

Linker 5

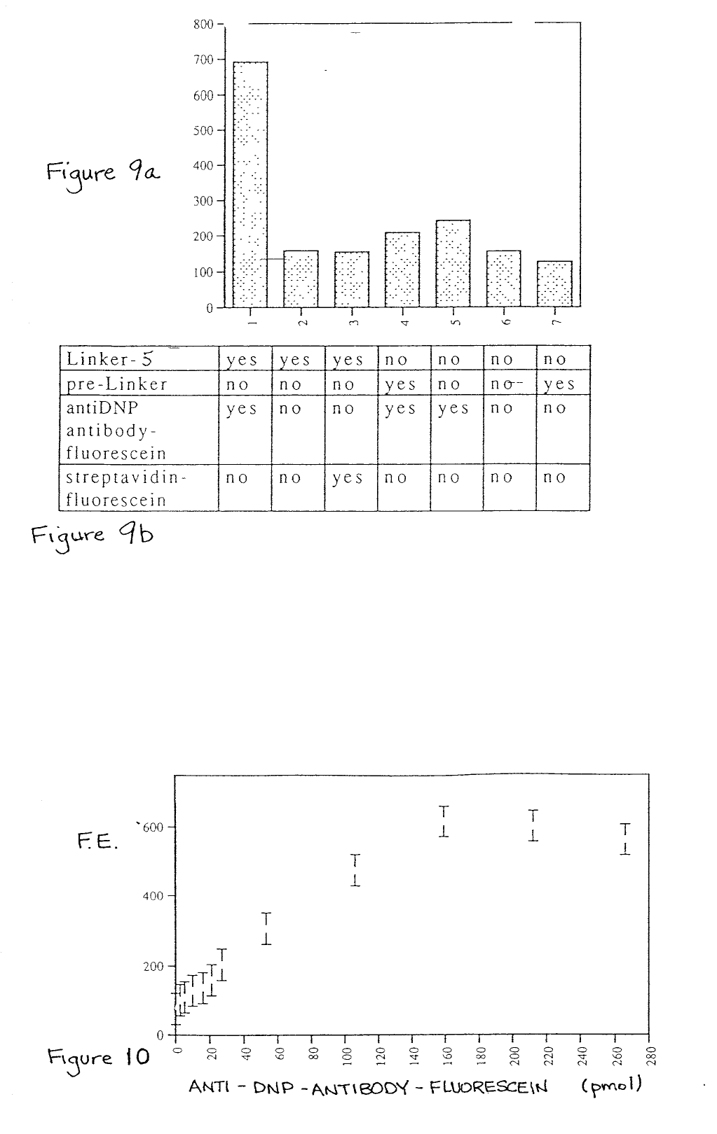

Experimental examination of linker 5 was also conducted following the same experimental procedure applied to linker 3 with the exception that fluorescein-anti-DNP (rabbit IgG fraction, fluorescein conjugate, Cat# A-6423, Molecular Probes, Inc., Eugene, Oreg.) replaced the fluorescein-streptavidin of the previous experiments. FIG. 9a shows the plot of fluorescence emission for the reaction conditions listed in FIG. 9b. The numerical data seen in Table 9 and the plot of corresponding FIG. 10 show fluorescence emission when 50 nmol of linker 5 was used.

TABLE 9

| | | [α-DNP] |
|---|---|---|
| | Column 1 | pmol |
| Row 1 | 561.46 | 266.0 |
| Row 2 | 599.48 | 212.0 |
| Row 3 | 615.29 | 159.0 |
| Row 4 | 473.57 | 106.0 |
| Row 5 | 306.3 | 53.0 |
| Row 6 | 204.54 | 27.0 |
| Row 7 | 160.48 | 21.0 |
| Row 8 | 137.9 | 16.0 |
| Row 9 | 129.19 | 10.0 |
| Row 10 | 109.82 | 5.3 |
| Row 11 | 101.45 | 2.6 |
| Row 12 | 76.392 | 0 |

Similarly, the concentration dependence of linker 5 was examined while the concentration of fluorescein-anti-DNP (266 pmol) was kept unchanged as shown by the data in Table 10 and the plot of the corresponding FIG. 11.

TABLE 10

| | | [5] |
|---|---|---|
| | Column 1 | nmol |
| Row 1 | 543.9 | 5000.0 |
| Row 2 | 516.83 | 4000.0 |
| Row 3 | 369.58 | 3000.0 |
| Row 4 | 351.7 | 2000.0 |
| Row 5 | 211.7 | 1000.0 |
| Row 6 | 224.27 | 500.0 |
| Row 7 | 225.98 | 400.0 |
| Row 8 | 180.98 | 300.0 |
| Row 9 | 172.66 | 200.0 |
| Row 10 | 176.22 | 100.0 |
| Row 11 | 197.62 | 50.0 |
| Row 12 | 208.43 | 0 |

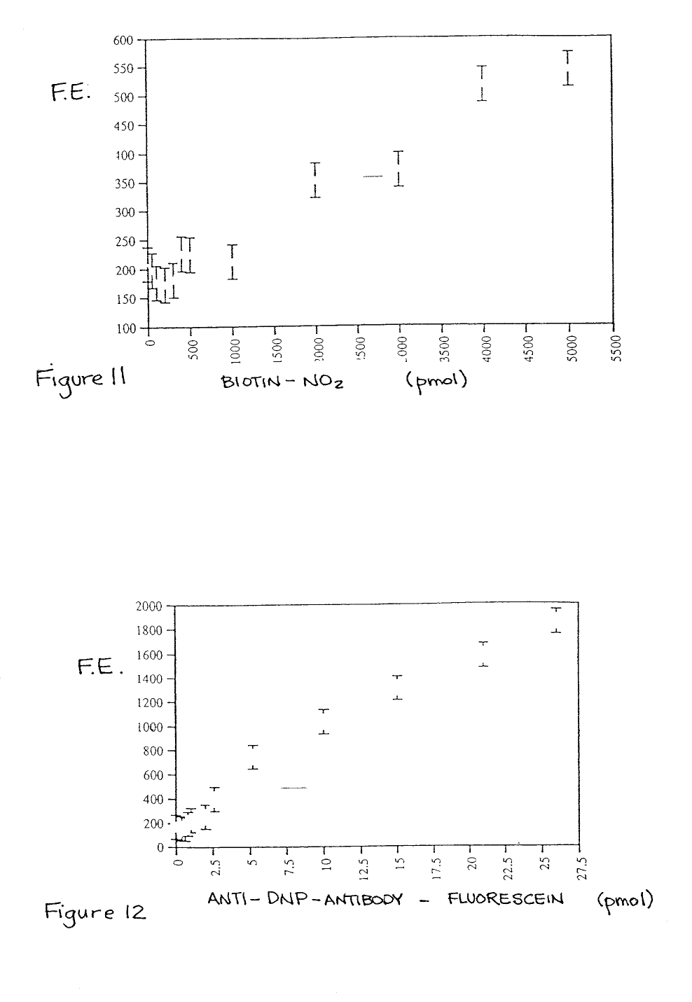

Table 11 shows a listing of the numerical data and the corresponding FIG. 12 illustrates the resulting fluorescence emission standard curve , using linker 5, which can be used to approximate the amount of fluorescein-protein binding to the linkers.

TABLE 11

| | | | | | | | [α-DNP] |
|---|---|---|---|---|---|---|---|
| | Column 1 | Column 2 | Column 3 | Column 4 | Column 5 | Column 6 | pmol |
| Row 1 | 1926.7 | 1821.8 | 1816.8 | 3748.5 | 5565.3 | 1855.1 | 26.0 |
| Row 2 | 1585.5 | 1617.7 | 1529.8 | 3203.2 | 4733.0 | 1577.7 | 21.0 |
| Row 3 | 1307.0 | 1296.1 | 1321.2 | 2603.1 | 3924.3 | 1308.1 | 15.0 |
| Row 4 | 1039.1 | 1034.7 | 1033.3 | 2073.8 | 3107.1 | 1035.7 | 10.0 |
| Row 5 | 771.5 | 721.95 | 749.02 | 1493.5 | 2242.5 | 747.49 | 5.2 |
| Row 6 | 386.93 | 449.81 | 352.65 | 836.74 | 1189.4 | 396.46 | 2.6 |
| Row 7 | 280.19 | 260.63 | 212.4 | 540.82 | 753.22 | 251.07 | 2.0 |
| Row 8 | 261.37 | 207.03 | 197.56 | 468.4 | 655.96 | 221.99 | 1.0 |
| Row 9 | 213.49 | 197.88 | 162.31 | 411.37 | 573.68 | 191.23 | 0.8 |
| Row 10 | 163.92 | 134.42 | 151.85 | 298.34 | 450.19 | 150.06 | 0.6 |
| Row 11 | 131.07 | 136.96 | 194.17 | 268.03 | 462.2 | 154.07 | 0.4 |
| Row 12 | 133.56 | 155.45 | 213.51 | 289.01 | 502.52 | 167.51 | 0 |

Upon examination of the experimental results, it becomes evident that the inclusion of the linkers of the present invention, specifically linkers 3 and 5, significantly enhances the binding capacity of both fluorescein-streptavidin (as in the generic System 2) and fluorescein-anti-DNP (as in the generic System 1). Experimental data for the studies using linker 3 show only a slight concentration dependence for the linker. For example, a 100-fold increase in fluorescein-streptavidin concentration in reaction with linker 3 produces only a 2-fold increase in fluorescence emission (e.g., 0.15 pm=110.75; 15 pm=220.15). Additionally, a 100-fold increase in linker concentration produces no significant increase in fluorescence emission (e.g., 0.5 nmol=239.14; 50 nmol=226.70). At much higher concentrations of linker 3, a 100-fold increase in linker concentration produces only a 2-fold increase in fluorescence emission. These results indicate that linker 3, in reaction with a fluorescence bound protein, produce a fluorescence emission capable of being detected even when small amounts of linker or protein are present.

Similarly, but to a somewhat lesser extent, reactions using linker 5 and fluorescein-antiDNP antibody show enhanced fluorescence emission. In experiments where the fluorescein-anti-DNP concentration is varied, a 10-fold increase in fluorescein-anti-DNP concentration produces only an approximately 3-fold increase in the amount of fluorescence emission (e.g., 5.3 pmol=109.82; 53 pmol=306.3). A 100-fold increase in linker 5 concentration in the reaction produces only a 2.5-fold increase in fluorescence emission. As with linker 3, linker 5 enhances the binding capacity of the fluorescein bound protein such that only small amounts of sample are needed for reaction detection. In a research environment where test samples are often available only in small quantities, the streptavidin array of the present invention maximizes the number of reactions capable of being run with minimum amounts of sample.

It will be apparent to those skilled in the art that various modifications and variations may be made in the system and method of the present invention without departing from the spirit or scope of the invention. Thus it is intended that the present invention cover the modification and variations of this invention provided they come within the scope of the appended claims and their equivalents.

We claim:

1. A screening system for small molecule-protein interactions comprising:
coating a solid phase support;
conjugating each small molecule library member with biotin through a tether;
affixing the small molecule library member to the solid phase;
interacting the affixed small molecule library member with a stock solution of protein containing a detectable tag bound to the protein; and
identifying the protein bound to the solid phase using a detector.

2. The screening system of claim 1, wherein said coating on the solid phase comprises streptavidin.

3. The screening system of claim 1, wherein said small molecule library members can be from the list consisting of enzyme inhibitors, peptides, biologically active compounds or small molecules.

4. The screening system of claim 1, wherein said stock solution of protein is any protein of known or unknown function.

5. The screening system of claim 1, wherein said detector is suitable for detecting the tag on the protein of known or unknown function.

6. The screening system of claim 1, wherein said tether is hydrophilic.

7. The screening system of claim 1, wherein the protein of known or unknown function is linked to the coated solid phase by binding the small molecule library member portion of the linker.

8. The screening system of claim 1, wherein said detectable tag is selected from the group consisting of radioactive tags and fluorescent dyes.

9. The screening system of claim 8, wherein said radioactive tag is selected from the group consisting of ³H, ³³P, ³⁵S and ¹²⁵I.

10. The screening system of claim 8, wherein said fluorescent tag is selected from the group consisting of fluorescein isothiocyanate, fluorescamine, rhodamine, Texas red, Alexa dyes and BODIPY dyes.

11. A method for preparing 2-dimensional arrays of tethered small molecules for use as protein affinity probes, the method comprising:
coating a solid phase support;
conjugating each small molecule library member with biotin through a tether;
affixing the small molecule library member to the solid phase;
interacting the affixed small molecule library member with a stock solution of protein containing a detectable tag bound to the protein; and
identifying the bound protein to the solid phase using an appropriate detector.

12. The method of claim 11, wherein said coating on the solid phase comprises streptavidin.

13. The method of claim 11, wherein said small molecule library members can be from the list consisting of enzyme inhibitors, peptides, biologically active compounds or small molecules.

14. The method of claim 11, wherein said stock solution of protein is any protein of known or unknown function.

15. The method of claim 11, wherein said detector is suitable for detecting the tag on the protein of known or unknown function.

16. The screening system of claim 11, wherein the protein of known or unknown function is linked to the coated solid phase by binding the small molecule library member portion of the linker.

17. The method of claim 11, wherein said tether is hydrophilic.

18. The method of claim 11, wherein said detectable tag is selected from the group consisting of radiolabeled tags and fluorescent dyes.

19. The screening system of claim 18, wherein said radioactive tag is selected from the group consisting of ³H, ³³P, ³⁵S and ¹²⁵I.

20. The method of claim 18, wherein said fluorescent tag is selected from the group consisting of fluorescein isothiocyanate fluorescamine, rhodamine, Texas red, Alexa dyes and BODIPY dyes.
